(19) United States
(12) Patent Application Publication
(10) Pub. No.: US 2013/0211345 A1
(43) Pub. Date: Aug. 15, 2013

(54) NEEDLE ASSEMBLY FOR DRUG DELIVERY DEVICES

(71) Applicants: Michael Jugl, Frankfurt am Main (DE); Axel Teucher, Frankfurt am Main (DE)

(72) Inventors: Michael Jugl, Frankfurt am Main (DE); Axel Teucher, Frankfurt am Main (DE)

(73) Assignee: SANOFI-AVENTIS DEUTSCHLAND GMBH, Frankfurt am Main (DE)

(21) Appl. No.: 13/698,021

(22) PCT Filed: May 17, 2011
(86) PCT No.: PCT/EP11/57957

§ 371 Date: Mar. 22, 2013

(30) Foreign Application Priority Data
May 18, 2010 (EP) 10163047.3

(51) Int. Cl. A61M 5/34

(52) U.S. Cl. CPC A61M 5/34
USPC 604/240; 29/428

(57) ABSTRACT

The present invention relates to a needle assembly for a drug delivery device comprising of a cup-shaped receptacle having a bottom section supporting a needle element being adapted to penetrate a pierceable seal of a cartridge to be disposed in a cartridge holder of the drug delivery device, wherein the receptacle comprises fastening means for releasably fastening the needle assembly to the cartridge holder, and wherein the fastening means is convertible into a release configuration by way of an elastic deformation of the receptacle.

### CROSS REFERENCE TO RELATED APPLICATIONS

The present application is a U.S. National Phase Application pursuant to 35 U.S.C. §371 of International Application No. PCT/EP2011/057957 filed May 17, 2011, which claims priority to European Patent Application No. 10163047.3 filed on May 18, 2010. The entire disclosure contents of these applications are herewith incorporated by reference into the present application.

### FIELD OF INVENTION

The present invention relates to a drug delivery device and in particular to a needle assembly to be interconnected to a cartridge holding section of a drug delivery device.

### BACKGROUND

Drug delivery devices allowing for multiple dosing of a required dosage of a liquid medicament, such as liquid drugs, and further providing administration of the liquid to a patient, are as such well-known in the art. Generally, such devices have substantially the same purpose as that of an ordinary syringe.

Such drug delivery devices have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reusable, the device should be inexpensive to manufacture and easy to dispose.

In order to meet these requirements, the number of parts and steps required to assemble the device and an overall number of material types the device is made from have to be kept to a minimum.

Typically, the medicament to be administered is provided in a cartridge that has a moveable piston or bung mechanically interacting with a piston rod of a drive mechanism of the drug delivery device. By applying thrust to the piston in distal direction, a certain amount of the medicinal fluid can be expelled from the cartridge.

Drug delivery devices, such like pen-type injectors typically comprise a housing having a cartridge holder for receiving the cartridge filled with the medicament that has to be dispensed. The distal end section of a cartridge holder facing towards the patient during an injector procedure typically comprises a through opening that provides access to a sealed distal end of the cartridge. By way of said through opening, an injection needle or cannula may penetrate the elastic seal to establish a fluid interconnect allowing the medicament to be expelled from the cartridge.

Typically, the disposable injection needle is provided by way of a needle assembly for releasably fastening the injection needle to the cartridge holder. For this purpose, needle assembly and distal end section of the cartridge holder comprise mutually corresponding threads, by way of which the needle assembly is screwed onto the cartridge holder. However, a threaded connection of cartridge holder and needle mount comes along with a number of deficiencies. Such a threaded engagement for instance, does not give a perceptible feedback to the user whether the needle assembly is securely mounted on the cartridge holder. In practical use, situations may occur, that only a single or a few threads of a cartridge holder and a needle assembly inter-engage, but the needle assembly is not yet securely fastened to the cartridge holder.

A user may then simply not be not aware of such improper and insufficient fastening and may therefore initiate a dose selecting and dispensing procedure. In particular in the course of a dispensing of a dose of the medicament, the needle assembly may autonomously disengage from the cartridge holder, for instance due to a built-up of a fluid pressure and/or when penetrating the skin of the patient. In these cases, the patient would be exposed to an increased health risk.

### SUMMARY

It is therefore an object of the present invention, to provide an improved fastening mechanism for a needle assembly and to provide an unambiguous mutual mechanical engagement of cartridge holder and needle assembly. In another object, the invention also aims to provide an easy and user-friendly way of releasably fastening a needle mount to a cartridge holder of a pen-type medical injection device. It is a further object, to provide a needle assembly mount which is beneficial with respect to production costs and which is particularly suitable for an industrial mass-production process.

In a first aspect, the invention provides a needle assembly for a drug delivery device, such as a pen-type injector for delivering multiple doses of a medicament, such like insulin. The drug delivery device typically comprises a housing and a drive mechanism to be operably engaged with a piston of a cartridge containing the medicament to be dispensed by the device. The housing comprises a cartridge holder or a cartridge holder section and may comprise multiple housing components, such as a main housing component adapted to accommodate the drive mechanism. The cartridge holder or the cartridge holder section is adapted to receive the cartridge.

Irrespective on whether the drug delivery device comprises a single- or multi-component housing, the cartridge holder or cartridge holder section at its distal end is adapted to receive and to support the needle assembly according to the present invention.

The needle assembly comprises a cup-shaped receptacle having a bottom section supporting a needle element. The needle element is adapted to penetrate a pierceable seal of the cartridge which is to be positioned in the cartridge holder. The needle element typically comprises a hypodermic needle. With its tipped end facing away from the cartridge, said needle element is adapted to penetrate biological tissue and in particular the skin of a patient for administering of the medicament.

The cup-shaped receptacle of the needle assembly comprises fastening means for releasably fastening the needle assembly to the cartridge holder. The fastening means according to the present invention and/or the receptacle itself is or are convertible into a release configuration, by way of an elastic deformation of the receptacle. Hence, by elastically deforming the receptacle, its fastening means can be transformed into a release configuration, in which the needle assembly can be removed from the cartridge holder.

Preferably, the receptacle or at least sections thereof, comprise a resiliently deformable material allowing to elastically deform the receptacle. The elasticity of the material and/or the receptacle is preferably chosen in such a way, that a user is able to initiate an elastic deformation, e.g. by squeezing the sidewalls of the receptacle with his fingers.

In a preferred embodiment, the fastening means is adapted to form a positive interlock with a corresponding fastening means of the cartridge holder. By way of a positive-engaged fastening of needle assembly and cartridge holder, an unambiguous fastening of the needle assembly can be provided. Needle assembly and cartridge holder are either released or inter-engaged, wherein both configurations are unambiguously recognizable by the end-user. By avoiding a gradual fastening means, such like a threaded connection of needle assembly and cartridge holder, a risk of autonomous disengaging of cartridge holder and needle assembly during dispensing of a dose can be minimized. Patient safety can therefore be improved compared to a conventional threaded engagement of needle assembly and cartridge holder.

In a further aspect, the fastening means is adapted to establish the positive interlock by displacing the cup-shaped receptacle translationally relative to the cartridge holder in a proximal direction. For mounting the needle assembly on a distal support section of the cartridge holder, the needle assembly is simply pushed onto the distal end section of the cartridge holder until mutually corresponding fastening means of needle assembly and cartridge holder inter-engage. Preferably, engagement of said fastening means is accompanied by some kind of visually and/or audibly perceivable signal, such like a clicking noise. Consequently, the user receives some kind of feedback, indicating that the needle assembly is securely fastened to the cartridge holder.

In a further preferred embodiment, the cup-shaped receptacle is oval or elliptical in cross section and comprises a resiliently deformable sidewall section. In typical application scenarios, the cup-shaped receptacle is initially oval, i.e. prior to a mounting onto the cartridge holder. In the course of mounting the needle assembly onto the cartridge holder, its cross section may become subject to modification due to mechanical interaction with the distal support section of the cartridge holder. The geometry of the cartridge holder may for instance be circular symmetric, thus enforcing or inducing the resilient deformation of the receptacle's cross section when mounted onto the cartridge holder.

In a further preferred aspect, the fastening means of the cup-shaped receptacle comprise at least two radially inwardly protruding latching elements that are arranged on opposite end points of an imaginary short axis of the ovally shaped receptacle. By having the latching elements disposed on the short side of the oval cross section of the receptacle, and by applying radially inwardly directed pressure on the long axis of the oval receptacle, the mutual distance of the latching elements can be increased. Due to such radially outwardly directed displacement, the latching elements of the receptacle and corresponding latching elements of the cartridge holder may disengage and the needle assembly can be detached and removed from the cartridge holder.

In still another embodiment, the latching elements comprise a bevelled face pointing towards a direction of assembly. When assembling needle assembly and cartridge holder, the bevelled face of the latching elements may support elastic deformation of the oval receptacle into a circular shape. Moreover, by way of the bevelled faces, a snap fit of the mutually corresponding latching elements can be attained. In effect, fastening of needle assembly and cartridge holder can be conducted in such a way, that the needle assembly is simply translationally displaced relative to the cartridge holder in proximal direction, i.e. towards the piston of the cartridge even without any twisting or turning motion.

In still another aspect the fastening means are convertible into a release configuration by applying radially inwardly directed pressure on opposite end points of an imaginary long axis of the oval receptacle. By way of exerting pressure across the oval's long axis, mutual distance between the latching elements disposed in the vicinity of the short axis can be increased for providing a decoupling and disengagement.

In a further aspect, the receptacle transforms or becomes deformed to a substantially circular symmetric cross section when converted into its release configuration. In its final assembly configuration at the cartridge holder, the receptacle returns to its substantially oval cross section while the cartridge holder is of substantially circular cross section. However, transformation of the cup-shaped receptacle into its release configuration may also be conducted in the course of assembly and may facilitate mutual engagement or snapping of corresponding fastening means of receptacle and cartridge holder.

In another independent aspect, the invention further relates to a cartridge holder subassembly for a drug delivery device. The subassembly comprises a cartridge holder adapted to receive a cartridge being filled with a medicament which is to be dispensed by the drug delivery device. The cartridge comprises a pierceable seal at a distal end portion and typically has a slidably disposed piston at the opposite, proximal end portion. By way of exerting distally directed pressure on the piston, a predefined amount of the medicament can be expelled from the cartridge if a fluid communication is established, e.g. by way of an injection needle penetrating the pierceable seal, e.g. the septum.

The cartridge holder further has a support for a needle assembly and further comprises fastening means adapted to form a positive interlock with corresponding fastening means of the above described needle assembly.

Preferably, the fastening means comprise an annular rim disposed at the outer circumference of the needle assembly support. Instead of a circumferential annular rim, it is also conceivable, that the fastening means comprise at least two or several radially extending ribs adapted to engage with radially inwardly protruding latching elements of the needle assembly.

In still another aspect, the support for the needle assembly comprises a substantially circular cross section, wherein oval or elliptical deviations from a circular cross section are still tolerable and are within the scope of the present invention. Preferably, the diameter of the circular cross section of the needle assembly support approximately equals the radial distance between latching elements of the needle assembly that are disposed at opposite sidewall sections of the receptacle, preferably overlapping with the imaginary short axis of the receptacle's oval cross section.

In a further independent aspect the invention also relates to a drug delivery device for administering at least one dose of a medicament. The device comprises a housing, a drive mechanism to be operably engaged with a piston of a cartridge for dispensing of a pre-defined dose of a medicament that is contained in said cartridge. The device further has a cartridge holder as well as a cartridge being displaced in the cartridge holder and being filled with the medicament. Additionally, the drug delivery device comprises a needle assembly as described above, which is adapted to be interconnected with the cartridge holder.

In still another aspect, the invention also provides a method of releasably fastening a needle assembly to a cartridge holder of a drug delivery device. The needle assembly comprises a cup-shaped and elastically deformable receptacle of substantially oval cross section and further comprises a bottom section supporting a needle element adapted to be pierced through a pierceable seal of a cartridge. Fastening of the needle assembly onto the cartridge holder comprises the steps of inserting a distal support of the cartridge holder of substantially circular cross section into the cup-shaped receptacle by displacing the receptacle translationally relative to the cartridge holder in proximal direction until radially inwardly protruding latching elements of the needle assembly establish a positive interlock with an annular rim disposed at the outer circumference of the distal support.

Preferably, releasable fastening of cartridge holder and needle assembly can be attained by exclusively conducting axial relative displacement of cartridge holder and needle assembly. Hence any twisting, screwing or rotational movements of needle assembly and cartridge holder is generally not required for fastening and assembling said components of a drug delivery device. A resilient deformation of the contour and shape of the needle assembly's receptacle is due to the geometric shape of the needle assembly support of the cartridge holder and its fastening means.

In a further preferred embodiment, disassembling of needle assembly and cartridge holder comprises the steps of applying radially inwardly directed pressure on opposite end points of an imaginary long axis of the ovally shaped cup-shaped receptacle for disengaging the positive interlock. Thereafter, needle assembly can be displaced translationally relative to the cartridge holder in axial and distal direction, hence away from the distal end of the cartridge holder.

The term “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
   wherein the group -Lys6-NH2 may be bound to the C-terminus of the Exendin-4 derivative;
   or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be apparent to those skilled in the pertinent art, that various modifications and variations can be made to the present invention without departing from the spirit and scope of the invention. Further, it is to be noted, that any reference signs used in the appended claims are not to be construed as limiting the scope of the present invention.

Without limitation, the present invention will be explained in greater detail below in connection with preferred embodiments and with reference to the drawings in which:

### BRIEF DESCRIPTION OF THE FIGURES

FIG. 1 schematically illustrates a cartridge holder subassembly with needle assembly attached thereto in cross sectional illustration,

FIG. 2 illustrates a simplified cross section through A-A according to FIG. 1 in a final assembly configuration and

FIG. 3 illustrates the cross section A-A according to FIG. 1 during disassembling of needle assembly and cartridge holder.

### DETAILED DESCRIPTION

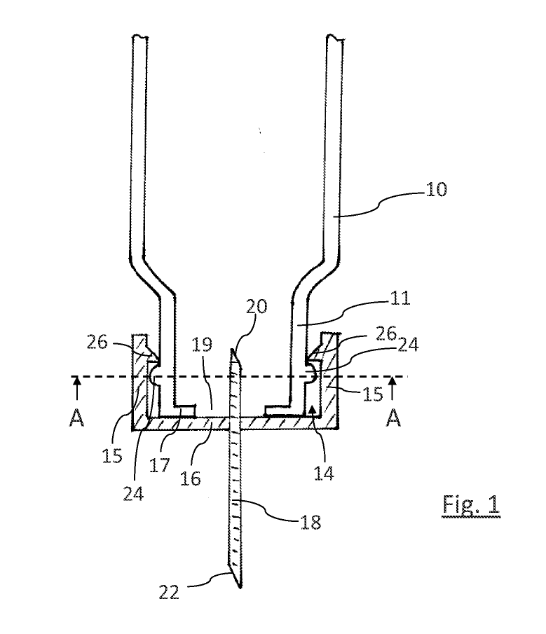

In FIG. 1, a cartridge holder subassembly is illustrated in a longitudinal cross section. The needle assembly 12 substantially comprises a cup-shaped receptacle 14 having a bottom section 16 and a substantially cylindrical and circumferential sidewall 15. In the centre of the bottom section 16, a piercing element, typically in form of an injection needle 18 is embedded having a distal tip 22 and a proximal tip 20. By way of the proximal tip 20, the needle 18 penetrates a pierceable septum of a cartridge, which is not explicitly illustrated here.

The cartridge holder 10 forms part of a housing of the not further illustrated drug delivery device. At its lower, distal section, the cartridge holder 10 comprises a neck portion 11, that serves as support for the cup-shaped receptacle 14.

At its distal end section, the cartridge holder 10 comprises a radially inwardly directed circumferential flange portion 17 abutting with the bottom section 16 of the needle assembly 12. Furthermore, radial extension of the flange 17 defines a through opening 19 for the proximal portion 20 of the needle 18.

As can be further seen from FIG. 1, needle assembly 12 and cartridge holder 10 are fastened to each other by way of inter-engaging fastening elements 24, 26. As illustrated in FIG. 1, the fastening means of the cartridge holder 10 is designed as radially protruding annular rim 24, whereas the corresponding fastening means of the cup-shaped receptacle 14 comprise radially inwardly protruding latching elements 26, that are adapted to form a snap fit with the annular rim 24 of the cartridge holder support 11.

As further illustrated in FIG. 1, radially inwardly protruding latching elements 26 comprise a bevelled surface, facilitating establishment of the snap fit as illustrated in FIG. 1. Hence, when the needle assembly 12 is simply translationally displaced relative to the cartridge holder 10 in proximal direction, which is upwards in FIG. 1, mutual interaction and inter-engagement of the corresponding fastening means 24, 26 will inherently lead to an at least temporary resilient deformation of the cross section of the cup-shaped receptacle 14 until the final assembly configuration is reached as illustrated in FIG. 1.

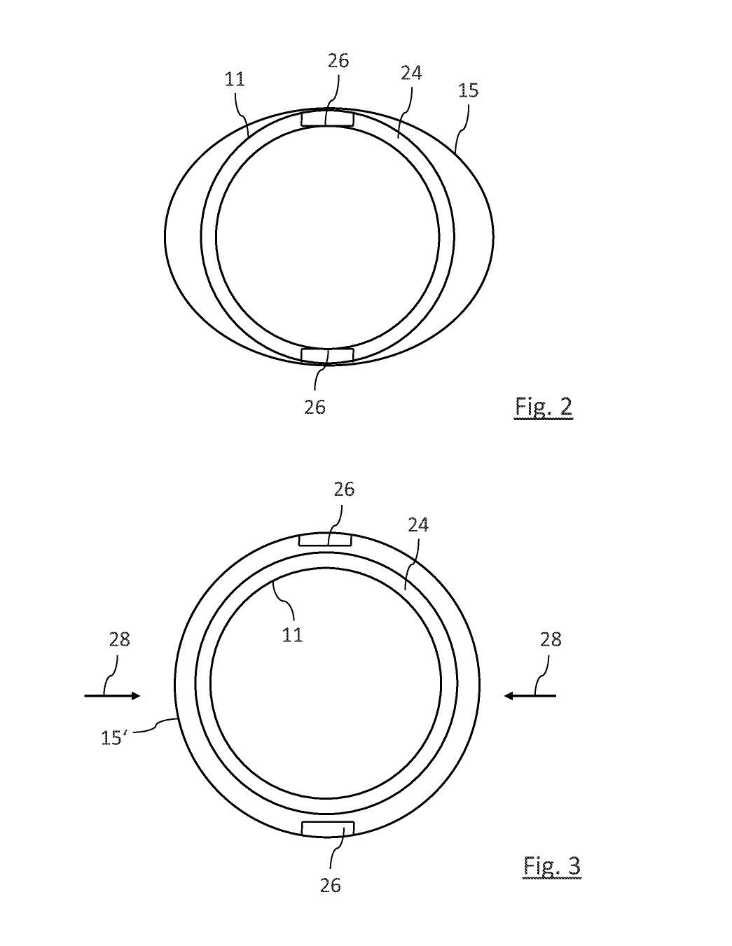

In FIGS. 2 and 3, a cross section of the cartridge holder 10 along A-A according to FIG. 1 is illustrated. The support section 11 is of rather circular symmetric geometry and its outer diameter is approximately in the range of the distance between oppositely disposed latching elements 26 of the receptacle 14 of the needle assembly 12. As illustrated in FIG. 2, radially inwardly protruding latching elements 26 of the needle assembly 12 geometrically overlap with the annular rim 24 of the cartridge holder 10. Since the latching elements 26 of the needle assembly 12 also abut in axial direction with the annular rim, the receptacle 14 and the needle assembly 12 are also fixed to the cartridge holder in axial direction.

In the opposite axial direction, mutual abutment of flange portion 17 and bottom section 16 limits any further displacement of the needle assembly 12 in proximal direction relative to the cartridge holder 10.

For releasing the snap fit as illustrated in FIG. 1, radially inwardly directed pressure 28 has to be applied to the sidewall 15 of the receptacle 14. Since the receptacle 14 is elastically or resiliently deformable, application of pressure 28 leads to a respective deformation of the cross sectional shape of the needle assembly 12. Hence, the side wall 15 becomes subject to a deformation 15′ and features a substantially circular cross section as depicted in FIG. 3.

Such a deformation inevitably comes along with an increase of the distance between the latching elements 26. The deformation-induced distance between opposite latching elements 26 becomes larger than the outer diameter of the annular rim 24. Consequently, inter-engagement of the mutually corresponding fastening means 24, 26 of needle assembly 12 and cartridge holder 10 is repealed and the needle assembly 12 is ready to be displaced in axial and distal direction relative to the cartridge holder 10.

Preferably, the receptacle 14 of the needle assembly 10 is manufactured as an injection molded piece and comprises elastomeric or polymeric material comprising a suitable degree of hardness or flexibility allowing for the intended elastic or resilient deformation of the receptacle 14 for the described releasable fastening to the cartridge holder 10.

1-12. (canceled)

13. A needle assembly for a drug delivery device comprising:
a cup-shaped receptacle having a bottom section supporting a needle element being adapted to penetrate a pierceable seal of a cartridge to be disposed in a cartridge holder of the drug delivery device,
wherein the receptacle comprises fastening means for releasably fastening the needle assembly to the cartridge holder,
wherein the fastening means is convertible into a release configuration by way of an elastic deformation of the receptacle, and
wherein the cup-shaped receptacle is initially oval in cross section and comprises a resiliently deformable side wall section,
wherein the fastening means comprise at least two radially inwardly protruding latching elements arranged on opposite endpoints of an imaginary short axis of the oval receptacle.

14. The needle assembly according to claim 13, wherein the fastening means is adapted to form a positive interlock with a corresponding fastening means of the cartridge holder.

15. The needle assembly according to claim 13, wherein the fastening means is adapted to establish the positive interlock by displacing the cup-shaped receptacle translationally relative to the cartridge holder in a proximal direction.

16. The needle assembly according to claim 13, wherein the latching elements comprise a bevelled face pointing towards a direction of assembly.

17. The needle assembly according to claim 13, wherein the fastening means are convertible into the release configuration by applying radially inwardly directed pressure on opposite end points of an imaginary long axis of the oval receptacle.

18. The needle assembly according to claim 13, wherein the receptacle transforms to a substantially circular symmetric cross section when converted into its release configuration.

19. A cartridge holder subassembly for a drug delivery device comprising
a cartridge holder adapted to receive a cartridge filled with a medicament to be dispensed by the drug delivery device, wherein the cartridge comprises a pierceable seal at a distal end portion,
a support for a needle assembly according to claim 13, and
fastening means adapted to form a positive interlock with corresponding fastening means of the needle assembly.

20. The cartridge holder subassembly according to claim 19, wherein the fastening means comprise an annular rim disposed at the outer circumference of the needle assembly support.

21. The cartridge holder subassembly according to claim 13, wherein the needle assembly support comprises a substantially circular cross section.

22. A drug delivery device for administering a dose of a medicament, comprising:
a housing,
a drive mechanism to be operably engaged with a piston of a cartridge for dispensing of a pre-defined dose of a medicament contained in said cartridge,
a cartridge holder,
a cartridge displaced in the cartridge holder and being filled with the medicament, and
a needle assembly according to claim 13.

23. A method of releasably fastening a needle assembly to a cartridge holder of a drug delivery device, wherein the needle assembly comprises a cup-shaped and elastically deformable receptacle of substantially oval cross section comprising a bottom section supporting a needle element, wherein fastening of the needle assembly to the cartridge holder comprises the steps of:
inserting a distal support of the cartridge holder of substantially circular cross section into the cup-shaped receptacle by displacing the receptacle translationally relative to the cartridge holder in proximal direction until radially inwardly protruding latching elements of the needle assembly form a positive interlock with an annular rim disposed at the outer circumference of the distal support.

24. The method according to claim 23, wherein disassembling of needle assembly and cartridge holder comprises the steps of:
applying radially inwardly directed pressure on opposite end points of an imaginary long axis of the oval shaped cup-shaped receptacle of the needle assembly for disengaging the positive interlock,
displacing the needle assembly translationally relative to the cartridge holder in distal direction away from the distal end of the cartridge holder.
